(19) United States
(12) Patent Application Publication
(10) Pub. No.: US 2016/0256104 A1
(43) Pub. Date: Sep. 8, 2016

(54) INDEPENDENT WEARABLE HEALTH MONITORING SYSTEM, ADAPTED TO INTERFACE WITH A TREATMENT DEVICE

(71) Applicant: HEALTHWATCH LTD., Herzliya (IL)

(72) Inventors: Yoram ROMEM, Herzliya (IL); Uri AMIR, Or Yehuda (IL)

(73) Assignee: HEALTHWATCH LTD., Herzliya (IL)

(21) Appl. No.: 15/030,318

(22) PCT Filed: Oct. 12, 2014
(86) PCT No.: PCT/IL2014/050895

§ 371 Date: Apr. 18, 2016

Related U.S. Application Data
(60) Provisional application No. 61/892,475, filed on Oct. 18, 2013.

(51) Int. Cl. A61B 5/00; A61B 5/0205; A61B 5/145; A61N 1/04; A61B 5/021; A61B 5/08; A61B 5/053; A61B 5/024; A61B 5/0402; A61B 5/04

(52) U.S. Cl. CPC A61B 5/4836; A61B 5/0402; A61B 5/6804; A61B 5/02055; A61B 5/14542; A61B 5/04012; A61B 5/746; A61B 5/021; A61B 5/0816; A61B 5/053; A61B 5/02438; A61B 5/4875; A61N 1/046; A61N 1/0484

(57) ABSTRACT

An independent wearable health monitoring system, configured for use by a living being on a daily basis. The system includes a knitted garment worn by the living being adjacently to preconfigured body locations, a garment-processing device having processor, and a multiplicity of sensors adapted to measure health parameters, wherein at least some sensors are integrally knitted with the knitted garment, and wherein the knitted textile sensors include electrodes adapted to provide ECG data. The system further includes an interface adapted to operatively connect at least one external medical device to the garment-processing device. Preferably, the health monitoring system further includes two conductive, integrally knitted pads operatively disposed tightly adjacently to the skin of the monitored living being, adapted to facilitate placing of a respective defibrillator paddles thereon and applying defibrillator shocks. Preferably, the garment-processing device controls the activation and deactivation of the defibrillator shocks.

### CROSS REFERENCE TO RELATED APPLICATIONS

This application claims the benefit under 35 USC 119(e) from U.S. provisional application 61/892,475 filed Oct. 18, 2013, the disclosure of which is included herein by reference.

This application also relates to PCT applications PCT/IL2010/000774 ('774), PCT/IL2012/000248 ('248), PCT/IL2013/050964 ('964) and PCT/IL2014/050134 ('134), the disclosures of which are included herein by reference in their entirety.

### FIELD OF THE INVENTION

The present invention relates to real-time health monitoring systems and more particularly, the present invention relates to an independent wearable, substantially continuous health monitoring system, carried by a monitored person (or another living being) by wearing special garments having textile electrodes knitted therein, such that the system does not interfere with the everyday life of the monitored living being. The system is adapted to plug-in external medical and other devices, such as a defibrillator, a team-tracker (sport, games, first-responders etc.) or an ergometer, that utilize ECG data or any other relevant system data such as blood pressure, oxygen saturation, breathing, temperature, dehydration, impedance and data obtained from any other sensors (and may be processed) that are in operative communicating with the controlling unit of the system.

Furthermore, the system of the present invention issues a personal-alert to the monitored person, and possibly to external parties, upon detecting a potentially health hazardous situation. The monitored person does not need to do anything in order to get a personal-alert, but just to wear the special garments (including an undershirt or a bra) that are part of the system and to have a smart device, such as his/her personal smartphone, nearby, thus facilitating the detection of a variety of health related abnormalities, including the main aspects of cardiac hazards such as Arrhythmia, Ischemia, heart failure and more.

### BACKGROUND OF THE INVENTION AND PRIOR ART

PCT application '774 explains the need for “a health monitoring system, implantable into and/or wearable by a living being to be monitored, wherein the system does not interfere with the everyday life of the monitored living being and issues an alert upon detecting a potentially health hazardous situation or a tendency to develop such situation.” Such an “early warning” system, is the basis of the current invention.

As described in '774, there is a need for a health monitoring system that continuously checks the well being of a person (or any other living being) that, typically, is considered healthy, or a person with a known set of diseases or a person in a specific risk group, covering a significant range of health hazards that may cause a significant life style change/limitation, and provides an alert as early as possible—all this, with no significant limitation to the normal life style of the person bearing the system. Naturally, such a system may also be used by a sick person, detecting potential exacerbations or new problems.

PCT application '964 describes a garment, having built-in sensors and electrodes, adapted to provide clinical level ECG, providing a 15-leads ECG, as well as measuring other health parameters.

There are other medical devices and systems that require heart parameters measurement to operate and that may take advantage of the clinical level ECG provided by the garment described in PCT application '964, for example a defibrillator.

In the case of a defibrillator, there is a need, in regular operation, to attach two paddles or pads to the chest of the person. This operation takes some critical time and needs to be performed by an experienced operator. Furthermore, the defibrillator usually needs to verify that the person is suitable to receive this treatment, by measuring ECG and determining whether the person has persistent ventricular fibrillation, a systole or consistence atrial fibrillation. This process, again, consumes critical time in the interval before being able to perform the actual defibrillation.

There is therefore a need and it would be advantageous to have an interface built into the garment described in PCT application '964, thereby adapting the garment to provide the data needed for the specific device or system interfacing into the garment. This will save critical time before the defibrillation.

There is also a need, and it would be advantageous to have built-in pads or paddles in the garment, to save additional critical time in their placement before the defibrillation.

In other cases, being able to track the person, including the motion/posture and the data from other sensors embedded or attached to the garment provides advantage in the gaming, exercising or emergency management.

The term “continuous monitoring”, as used herein with conjunction with a health monitoring system, refers to a health monitoring system, facilitated to monitor a living being substantially continuous, day and night, when the monitored living being is awake or asleep, and active in substantially all common activities of such living being.

The term “seamless”, as used herein with conjunction with a wearable device, refers to a device that when worn by an average person, wherein the device puts no significant limitation to the normal life style of that person and preferably not seen by anybody when used and not disturbingly felt by the user while wearing it. Furthermore, no activity is required from the monitored person in order for the system to provide a personal-alert when needed. It should be noted that people that pursue non-common life style, such as soldiers in combat zone or in combat training zone, or firefighters in training and action, or athletes in training or competition may utilize non-seamless devices. As the “seamless” characteristics refers also to the user's behavior, the wearable component is preferably an item that is normally worn (e.g., underwear) and not some additional item to be worn just for getting the alert.

The terms “underwear” or “garment”, as used herein with conjunction with wearable clothing items, refers to seamless wearable clothing items that preferably, can be tightly worn adjacently to the body of a monitored living being, typically adjacently to the skin, including undershirts, brassiere, underpants, socks and the like. Typically, the terms “underwear” or “garment” refer to a clothing item that is worn adjacently to the external surface of the user's body, under external clothing or as the only clothing, in such way that the fact that there are sensors embedded therein and/or integrated therein, is not seen by any other person in regular daily behavior. An underwear item may also include a clothing item that is not underwear per se, but still is in direct and preferably tight contact with the skin, such as a T-shirt, sleeveless or sleeved shirts, sport-bra, tights, dancing-wear, and pants. The sensors, in such a case, can be embedded in such a way that are still unseen by external people to comply with the “seamless” requirement.

The term “tightly” means that specific portions of the garment where there are electrodes or other sensors that require certain pressure on the body to obtain a satisfactory signal, are designed to be as tight as needed. However, all the other parts of the garment may be not as tight. Optionally, there is a provision to facilitate tightening or releasing certain portions of the garment, by built-in straps or other tightening means, so that the need for more or less tightness does not require the replacement of the whole garment.

The term “independent” as used herein with conjunction with a garment, having a wearable device or a health monitoring system, refers to an item that does not depend on any external entity, such as remote monitoring center, but may operationally depend on another regular common personal item of the same user, such as a personal mobile device having a garment-processing application, being part of the health monitoring system. It should be noted that if the monitored living being is not a human being, the personal mobile device is carried by the care-taker of the monitored living being. It should be noted that in the case of an animal, the seamless feature is of lesser importance.

The term “abnormal”, as used herein with conjunction with health related parameters, refers to a parameter value or one or more ranges of values which are defined as health hazardous or as potential health hazardous, when a trend is identified, and requires attention. For example, the normal blood pressure of an adult person is in the range 120/80 mm Hg. Typically, a systolic blood pressure of 130 mm Hg would not be considered hazardous. However, if a person has a stable mean blood pressure of around 85±10 mm Hg, and suddenly it increases to 125±10 mm Hg, this may be considered as an abnormal situation. Likewise, if the mean blood pressure changes gradually and consistently from 85 mm Hg to 120 mm Hg, in a clear trend, a personal-alert should be issued. The threshold value from which the high blood pressure parameter is considered as health hazardous may vary and can be set personally and optionally, dynamically updated, either manually or automatically, by an adaptation algorithm. Once the high blood pressure parameter, in the above example, is set, any value out of the set threshold value will then be considered as abnormal for that person.

The phrase “clinical level ECG”, as used herein with conjunction with ECG measurements, refers to the professionally acceptable number of leads, sensitivity and specificity needed for a definite conclusion by most cardiology physicians to suspect a risky cardiac problem (for example, arrhythmia, myocardial ischemia, heart failure) that require immediate further investigation or intervention. Clinical level ECG is derived from a pro-configured number of ECG leads, typically, with no limitations 12 and up to 15-leads ECG, and further preferably coupled with a motion/posture compensation element, and a real-time processor with adequate algorithms.

The term “personal-alert”, as used herein, is a notification issued to the specific user or a designated person (e.g., a person responsible to perform medical intervention in emergency situations or perform the defibrillation), after detecting a health risk hazard by a system according to the present invention. The personal-alert issued by the system is substantially similar to a decision of a family doctor or another professional physician who would have taken, seeing the abnormal measured parameters, in order to instruct further investigation or intervention. The term “specific user” as used herein, means that the personal-alert decision is preferably made while taking into consideration the history of indications, treatments and personal situation of the user, including personal preferences and other personally adapted considerations.

A number of systems that analyze a cardiac patient's condition are commonly used. Such systems include some form of ECG electrodes (“probes”) that are removably attached adjacent to the patient's body and are connected to the system. Typically, the electrodes are securely attached to the patient's body at a selected location by suction cups, pads having two-sided glue and other attaching means that can be securely attached to the patient's body and forcedly removed when the measurements are concluded. Thereby, the electrode remains attached to the patient's body at a specific location during the time of measurement, which time is very limited and typically lasts a few minutes up to a few hours.

Also, typically, either a physician or a nurse is responsible for the actual placement of the electrodes at the specific points known to be adequate for accurate ECG measurements.

However, when using a wearable, continuous real-time health monitoring system, worn by a monitored person thereby placing the probes just by wearing, a sensor that is built into garment remains in the vicinity of a specific target bodily location of the monitored person. However, still, there are some changes in the relative position of the sensor with respect to a specific bodily target location of the monitored patient, due to relative movements of the garment, carrying the sensor, with respect to the body of the monitored patient, or due to the other reasons.

There is therefore an additional need to ensure a reliable sensed signal from a probe, integrated into a garment, requiring at least some compensation for both an initial misplacement and physical activity displacement.

It should be noted that such a mechanism can be useful in any system with sensors and probes that may move away from their optimal location, thus degrading the quality of their measurement.

It should be further noted that there are several levels of alerts, and several levels require some operation of interfaced devices, such as, with no limitations, activation and deactivation of a defibrillator.

### BRIEF SUMMARY OF THE INVENTION

The principal intentions of the present invention include providing a health-monitoring and self-alert system, including a smart garment adapted to issues a personal-alert to the monitored person wearing the garment (and possibly to external parties), upon detecting a potentially health hazardous situation, including a cardiac related health hazardous situation. The monitored person does not need to do anything in order to get a personal-alert, but just to wear the smart garment (including an undershirt or a bra) that are part of the system, and to have a smart device, such as his/her personal smart-phone, nearby. A variety of sensors are embedded and/or integrated into the smart garment, thus enabling the detection of a variety of health related abnormalities, including the main aspects of cardiac hazards such as Arrhythmia, Ischemia, heart failure and more. The sensors include textile electrodes for measuring clinical level ECG, providing up to (with no limitations) 15-leads ECG.

The system of the present invention is adapted to measure health related physiological bodily parameters, including (with no limitations) measuring preferably clinical level ECG, analyzing the data in real-time, and upon detecting a situation which requires further investigation or immediate intervention, alarms the system carrier to seek medical help. Furthermore, the wearable system includes an interface adapted to operatively connect to one or more external medical systems or devices, such as, with no limitations, a defibrillator, and to provide required data and analysis such as persistent ventricular fibrillation or ventricular tachycardia indications.

The present invention will be described in terms of the external interfacing device being a defibrillator, but the present invention is not limited to interfacing to a defibrillator, wherein the external interfacing device or system can be any device/system that needs the ECG data to operate properly, for example an ergometer.

According to the teachings of the present invention, there is provided an independent wearable health monitoring system, configured for use by a living being on a daily basis, including a healthy living being. The system includes a knitted garment worn by the living being adjacently to preconfigured portions of the body of the living being, a garment-processing device and a multiplicity of sensors adapted to measure health parameters, wherein at least a portion of the sensors are integrally knitted with the knitted garment, and wherein the knitted textile sensors include electrodes or probe-devices adapted to provide ECG data. The system further includes an interface adapted to operatively connect at least one external medical device to the garment-processing device. The garment-processing device includes a garment-processor and a power sources such as a battery.

The garment-processing device is adapted to acquire the ECG data from the sensors and analyze the ECG data, and upon detecting at least one predefined abnormality in the ECG data, activating at least one pre-selected the external medical device configured to cope with the detected abnormality.

Optionally, the health monitoring system further includes an alerting unit, wherein the garment-processing device, upon detecting at least one predefined abnormality in the ECG data, activates the alerting unit. Optionally, the ECG data in a clinical level ECG data.

Optionally, the sensors include sensors for sensing blood pressure, oxygen saturation, breathing, temperature, dehydration, impedance, sweat analysis, lung fluids and heart rate.

Optionally, the health monitoring system is seamless self-alert system.

In some embodiments, the health monitoring system further includes at least one conductive knitted pad operatively disposed tightly adjacently to the skin of the monitored living being, wherein the at least one conductive knitted pad is adapted to facilitate placing of a respective defibrillator paddle thereon and applying defibrillator shocks. Preferably, the garment-processing device is operatively connected to an apparatus controlling the at least one defibrillator paddle, and wherein the garment-processing device controls the activation and deactivation of the defibrillator shocks. Optionally, the at least one defibrillator paddle is manually activated and deactivated.

Optionally, the knitted garment has a tubular form having variable elasticity, and wherein the at least one conductive knitted pad is operatively disposed tightly adjacently to the skin of the monitored living being.

Optionally, the garment-processing device further includes protecting means to protect the garment-processing device from the current surge inflicted by the defibrillator onto the at least one conductive knitted pad.

### BRIEF DESCRIPTION OF THE DRAWINGS

The present invention will become fully understood from the detailed description given herein below and the accompanying drawings, which are given by way of illustration and example only and thus not limitative of the present invention:

FIG. 1 (prior art) schematically illustrates a seamless independent wearable health monitoring and self-alert system, including a knitted garment-body being an undershirt, interconnected sensors/electrodes by conductive yarns, a garment-processing device and a mobile device having a remote-processor, as described PCT application '964.

FIG. 2 is a schematic illustration of the undershirt shown in FIG. 1, adapted to operatively interface with an external device, for example a defibrillator, according to embodiments of the present invention, wherein the defibrillator paddles are placed over respective textile pads integrally knitted into the undershirt.

FIG. 3a is a schematic illustration the undershirt shown in FIG. 2, having the defibrillator paddles removed.

FIG. 3b is a detailed view illustration of window A, shown in FIG. 3a.

FIG. 4a depicts a front view of an exemplary garment of the schematic system shown in FIG. 3a, wherein the textile electrodes are designed to measure a 15-lead ECG signal.

FIG. 4b depicts a side view of the garment shown in FIG. 4a.

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein; rather, these embodiments are provided, so that this disclosure will be thorough and complete, and will fully convey the scope of the invention to those skilled in the art.

An embodiment is an example or implementation of the inventions. The various appearances of “one embodiment,” “an embodiment” or “some embodiments” do not necessarily all refer to the same embodiments. Although various features of the invention may be described in the context of a single embodiment, the features may also be provided separately or in any suitable combination. Conversely, although the invention may be described herein in the context of separate embodiments for clarity, the invention may also be implemented in a single embodiment.

Reference in the specification to “one embodiment”, “an embodiment”, “some embodiments” or “other embodiments” means that a particular feature, structure, or characteristic described in connection with the embodiments is included in at least one embodiments, but not necessarily all embodiments, of the inventions. It is understood that the phraseology and terminology employed herein is not to be construed as limiting and are for descriptive purpose only.

Meanings of technical and scientific terms used herein are to be commonly understood as to which the invention belongs, unless otherwise defined. The present invention can be implemented in the testing or practice with methods and materials equivalent or similar to those described herein.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

It should be noted that the present invention will often be described in terms of the knitted monitoring-garment being an undershirt, but the present invention is not limited to an undershirt being the monitoring-garment, and type of garment, at least partially worn adjacently to the body of the monitored living being can be used as a monitoring-garment.

It should be noted that the present invention will be described in terms of the mobile device being a smart-phone, but the present invention is not limited to being a smart-phone, and includes all types of mobile devices having a central processing unit and memory, including a mobile phone, laptop, a PDA, a processing pad, etc., all having

Bluetooth or any other wireless communication capabilities. According to the teachings of the present invention, there is provided an independent, seamless and preferably substantially continuous health monitoring system, designed for use by a healthy living being but also suitable for non-healthy living being.

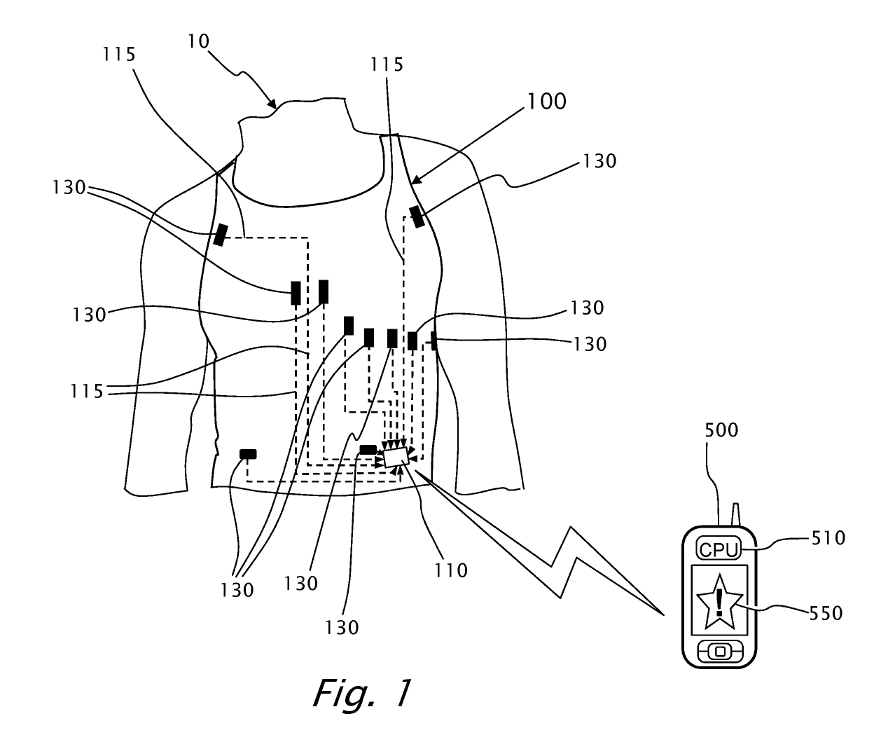

Reference now made to the drawings. FIG. 1 (prior art) schematically illustrates an example seamless, independent, wearable and preferably continuous health monitoring and self-alert system 100 (also referred to herein as “health-monitoring-garment-based system 100”), including a basic knitted garment 102 having a tubular form, a removable garment-processor 110 and optionally, a mobile device 500, having a remote-processor 510. Health-monitoring-garment-based system 100 is a non-limiting, exemplary monitoring-garment item, wherein sensors 130 are operatively with garment-processing device 110, as schematically illustrated in FIG. 1. Sensors 130 are embedded into the garment-body 102 of monitoring-garment 100, preferably knitted therein. Preferably, sensors 130 are formed by washable yarn, when the garment is knitted.

Typically, health-monitoring-garment-based system 100 looks like a regular undershirt and preferably, the embedded sensors are adapted to sense physiological bodily parameters, including (with no limitations) measuring cardiac related parameter such ECG . A person 10 can easily wear the undershirt in any situation where he or she is used to. When the undershirt is firstly provided to person 10, the size and tightness to the person's body are fitted such that sensors 130 are disposed at the correct bodily regional places and with appropriate contact with the person's body, tightened to the skin. Tightness is achieved by using preconfigured variable elasticity of knitted garment 102, as described in '134.

Health-monitoring-garment-based system 100 may include a variety of sensors, including optical sensors for sensing the oxygen level in the blood. ECG sensors are for detecting heart-rate (HR) related irregularities (arrhythmia) as well as ischemia (more precisely, placed at the standard ECG positions), facilitated by up to 15-leads ECG (with no limitations). Acoustic sensors are for detecting lung fluids and HR and impedance sensors are for detecting congestive heart failure (CHF). Also, breathing sensors, such as carbon-elastomer stretch or impedance sensors that can detect breathing rhythm and breathing regularity or irregularity. A pressure sensor is an example sensor that measures the in and out motion of the thorax, facilitating detecting breathing and measuring breathing rate. The sensors may further include sweat analysis sensors, temperature and other sensors.

Knitted garment 102 of health-monitoring-garment-based system 100 is preconfigured for wear either by a man or a woman and preferably, comes in a variety of sizes.

Preferably, also embedded into knitted garment 102 are conductive textile wires 115, connecting the sensors to garment-processor 110. Optionally, also embedded into knitted garment 102 are wires interconnecting some of the sensors (e.g. between couples of impedance sensors). In some embodiments, conductive textile wires 115 are connected to garment-processor 110 via click-on button interface 111. In some embodiments, conductive textile wires 115 are connected to garment-processor 110 via an HDMI interface. Such a connecting mechanism is described in U.S. Provisional Patent Applications 61/981,213 filed in Apr. 18, 2014 and 62/014,753 filed in Jun. 20, 2014, the disclosures of which are included herein by reference in their entirety.

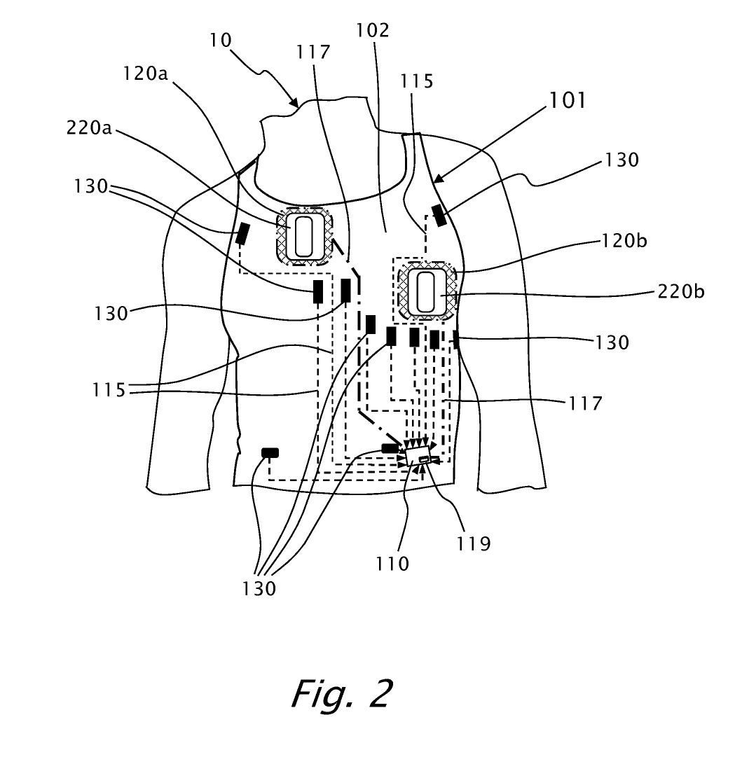

Reference is now made to FIG. 2, schematically illustrating a health-monitoring-garment-based system 101, which health-monitoring-garment-based system 101 is similar to health-monitoring-garment-based system 100, wherein knitted garment 102 may further includes conductive pads 220 integrally knitted into knitted garment 102. Hosting pads 220, being operatively disposed tightly adjacently to the skin of person 10, are adapted for hosting paddles 220 of a defibrillator, wherein hosting pads 220 are connected to garment-processing device 110 by designated conductive wiring 117, such as conductive yarns, according to embodiments of the present invention. The paddles of a defibrillator may also be attached to knitted garment 102 at selected locations by any other attaching mechanism known in the art. The paddle, however, are connected to garment-processing device 110 by designated conductive wiring 117, according to other embodiments of the present invention.

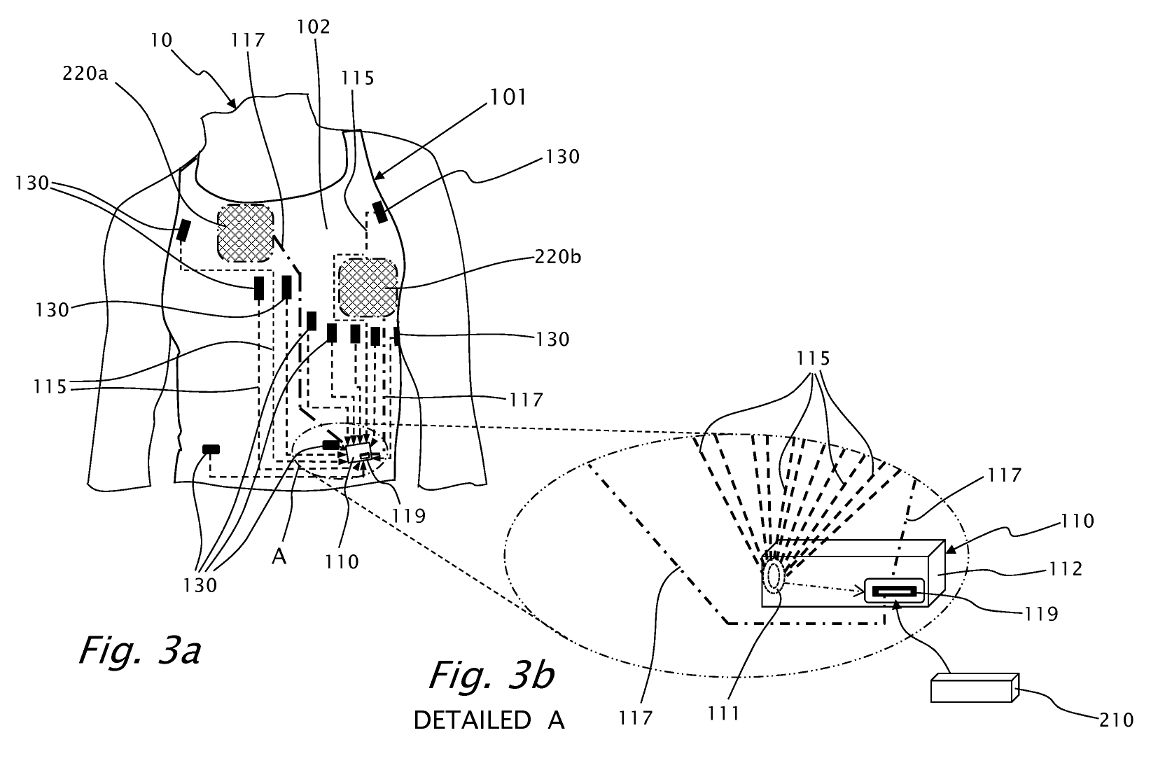

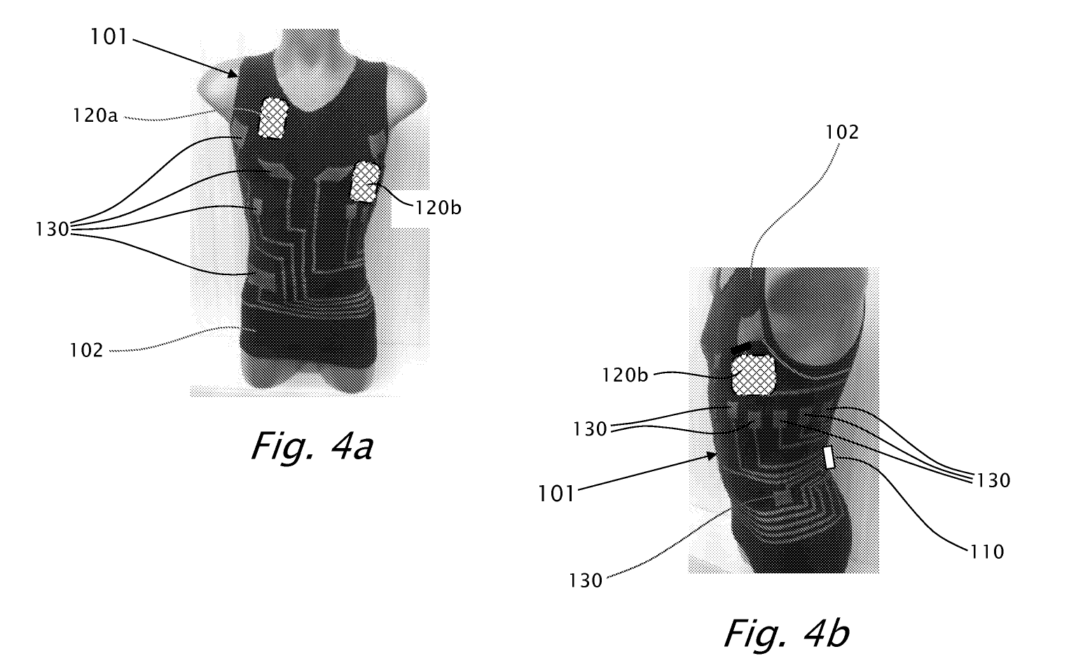

Reference is also made to FIG. 3a, schematically illustrating a health-monitoring-garment-based system 101, as schematically shown in FIG. 2, wherein the defibrillator paddles 220 are removed from hosting paddles 220. FIG. 4a depicts a front view of an exemplary garment 101 of the schematic system shown in FIG. 3a, wherein the textile electrodes are designed to measure up to 15-lead ECG signal. FIG. 4b depicts a side view of garment 101 shown in FIG. 4a.

Reference is also made to FIG. 3b, a detailed view illustration of a window A shown in FIG. 3a that illustrates health-monitoring-garment-based system 101, wherein garment-control device 110 further includes an interface 119 adapted to interconnect with an external device 210 or an interface thereof.

Interface 119 is adapted to transfer relevant system data to one or more external medical systems or devices such as a defibrillator, operatively connect to health-monitoring-garment-based system 100 via an interface such as interface 119. In the case of a defibrillator, health-monitoring-garment-based system 100 provides required data and analysis such as persistent ventricular fibrillation. Optionally, the external device is given permission to control usage of the embedded sensors and other usable devices of health-monitoring-garment-based system 100. Optionally, interface 119 is physically separated from garment-processing device 110. In the case of a defibrillator, interface 119 is designed to withstand high electric current surges.

In the case external device 210 is a defibrillator, hosting pads 220 are operatively connected to garment-processing device 110 by designated conductive wiring 117. Garment-processing device 110 further includes an interface 119 adapted to interconnect with the external defibrillator (or any other external device), wherein external device 210 is operatively connected to the paddles held by hosting pads 220. Thereby, when garment-processing device 110 determines a pre-determined health related situation that requires activation of the defibrillator paddles, garment-processing device 110 activates external device 210 to thereby activate the paddles via interface 119. Upon detecting cardiac vitality, garment-processing device 110 deactivates external device 210 to thereby deactivate the paddles.

Alternatively, a preconfigured number of designated textile electrodes, disposed at preconfigured location, about the heart position, may be activated manually by a medical personal, by connecting directly to the designated textile electrodes.

Since pads 220 and sensors 130 must be in substantially adjacent to the user's skin, pads 220 and the sensors 130 are typically spatially separated on knitted garment 102. However, since the contact surface of a pad 220 is substantially larger than that of a sensor 130, one or more sensors 130 may be disposed in a void formed inside a pad 220, or may be part of a pad 220, provided that sensor 130 is immune to high electric surges.

Optionally, the external defibrillator is removably connected to a designated button, similar to button 111, wherein optionally, the designated button facilitates a click-on connection or removal operations, and wherein the designated button is operatively connected to the processor of garment-processing device 110.

In some embodiments of the present invention, garment-processing device 110 analyzes the sensed data obtained by one or more of the sensors 130 to thereby determine if a health hazardous situation has occurred. In such an event, garment-processing device 110 activates an alerting unit, coupled to operate with garment-processing device 110, to thereby provide a personal-alert to person 10. The personal-alert may be in the form of an audio sound, a light indication, any other form known in the art, or a combination thereof.

Garment-processing device 110 may further calculate values, compare thresholds, trends, averages etc., and may provide the calculated data to an external recipient. Optionally, garment-processing device 110 further includes memory for storing data for calculations, comparisons to past measurements, determining trends, calibration, determining sensors reliability, further remote analysis at external places and for future use (for example, for use in physical exercise consulting).

In some embodiments of the present invention, garment-processing device 110 is subdivided into multiple, individual processing units, wherein each of the individual processing unit is operatively coupled with one or more sensors.

Optionally, garment-processor 112 does not have an “On/Off” button, but is activated automatically when engaged with button 111. This eliminates the need of a manual activation by the user, which is a source for errors and inconvenience.

To facilitate clinical level ECG measurements and thereby clinical level cardiologic ischemia analysis, ECG sensors 130 may be configured as multiple-lead ECG, preferably 12 or 15-leads ECG, as shown in FIG. 1, having additional electrodes on the back (such as at positions V₇, V₈) and on the left side of person 10. For activating a defibrillator, the only sensors that the health monitoring and self-alert system needs are the multiple-lead ECG (multi-lead ECG), facilitating clinical level ECG measurements and thereby clinical level cardiologic ischemia analysis. The ECG can thus be a 15-leads ECG (for added sensitivity), an 18-lead ECG or any additional number of electrodes that the wearable platform enables. The sensors are embedded in the monitoring-garment so that they are tightened to the skin at a respective preconfigured position, per each sensor's specific functionality (e.g. ECG—standard positions, acoustic—at the basal aspects of the lungs).

The ECG can detect, for example, HR related irregularities as well as ischemia (for example, ST elevation and depression, T-wave inversion and new left bundle branch block). Blood pressure is also indicative of heart or other cardio-vascular problems, as well as body temperature changes.

In some embodiments of the present invention the interface by which interface conductive textile wires 115 are connected to garment-processor 110, such as an HDMI interface, includes a protecting means to protect garment-processor 110 from the current surge inflicted by the defibrillator. The protecting means may include Zener diodes and or other current surge protecting means such as ZL70584 an eight-terminal IC, for example.

It should be noted that the pads for housing the defibrillator can be part of the wearable garment or external (attached defibrillator connecting to the control unit of the garment including external pads). In this configuration, time is saved in the process of getting all the data needed for the appropriate activation of the defibrillator, but the time needed to attach the defibrillator is not saved.

It should be further noted that the garment may have a special “emergency tearing” when there is a need for immediate defibrillation.

The invention being thus described in terms of embodiments and examples, it will be obvious that the same may be varied in many ways. Such variations are not to be regarded as a departure from the spirit and scope of the invention, and all such modifications as would be obvious to one skilled in the art are intended to be included within the scope of the following claims.

What is claimed is:

1. An independent wearable health monitoring system, configured for use by a living being on a daily basis, including a healthy living being, the system comprising:
a) a knitted garment worn by the living being adjacently to preconfigured portions of the body of the living being;
b) a garment-processing device comprising:
i) a garment-processor; and
ii) a battery;
c) a multiplicity of sensors adapted to measure health parameters, wherein at least a portion of said sensors are integrally knitted with said knitted garment, and wherein said knitted textile sensors include electrodes or probe-devices adapted to provide ECG data; and
d) an interface adapted to operatively connect at least one external medical device to said garment-processing device,
wherein said garment-processing device is adapted to acquire said ECG data from said sensors and analyze said ECG data, and upon detecting at least one predefined abnormality in said ECG data, activating at least one pre-selected said external medical device configured to cope with said detected abnormality.

2. A health monitoring system as in claim 1 further comprising an alerting unit, wherein said garment-processing device, upon detecting at least one predefined abnormality in said ECG data, activates said alerting unit.

3. A health monitoring system as in claim 2, wherein said ECG data in a clinical level ECG data.

4. A health monitoring system as in claim 1, wherein said sensors include sensors for sensing blood pressure, oxygen saturation, breathing, temperature, dehydration, impedance, sweat analysis, lung fluids and heart rate.

5. A health monitoring system as in claim 1, wherein the health monitoring system is seamless self-alert system.

6. A health monitoring system as in claim 1 further comprising at least one conductive knitted pad operatively disposed tightly adjacently to the skin of the monitored living being, wherein said at least one conductive knitted pad is adapted to facilitate placing of a respective defibrillator paddle thereon and applying defibrillator shocks.

7. A health monitoring system as in claim 6, wherein said garment-processing device is operatively connected to an apparatus controlling said at least one defibrillator paddle, and wherein said garment-processing device controls the activation and deactivation of said defibrillator shocks.

8. A health monitoring system as in claim 6, wherein said at least one defibrillator paddle is manually activated and deactivated.

9. A health monitoring system as in claim 6, wherein said knitted garment has a tubular form having variable elasticity, and wherein said at least one conductive knitted pad is operatively disposed tightly adjacently to the skin of the monitored living being.

10. A health monitoring system as in claim 6, wherein said garment-processing device further comprises protecting means to protect said garment-processing device from the current surge inflicted by the defibrillator onto said at least one conductive knitted pad.
